(19) United States
(12) Patent Application Publication
(10) Pub. No.: US 2024/0041151 A1
(43) Pub. Date: Feb. 8, 2024

(54) AUXILIARY DEVICE FOR SLEEPING ON SEAT

(71) Applicant: Jintao Liao, Shenzhen (CN)

(72) Inventor: Jintao Liao, Shenzhen (CN)

(21) Appl. No.: 18/381,632

(22) Filed: Oct. 18, 2023

(30) Foreign Application Priority Data
Aug. 16, 2023 (CN) 202322211871.5

(51) Int. Cl. A42B 3/12; A42B 3/14; A42B 3/16

(52) U.S. Cl. CPC A42B 3/125; A42B 3/147; A42B 3/16

(57) ABSTRACT

The present disclosure provides an auxiliary device for sleeping on a seat. The auxiliary device for sleeping on a seat includes a pillow. The pillow can be stably connected to a seat headrest through assembly parts. An eye mask is arranged on the side, away from the seat headrest, of the pillow. The eye mask has the function of isolating external light. Therefore, the rest quality of people is improved, and accidents can also be prevented. In addition, the eye mask is detachably connected with the pillow through the hook-and-loop fasteners, and the assembly parts can be quickly disassembled and assembled by using binding bands and connecting buckles, so that the using efficiency of the device is improved.

### TECHNICAL FIELD

The present disclosure relates to the technical field of auxiliary devices for seats, in particular to an auxiliary device for sleeping on a seat.

### BACKGROUND

It is pleasant to stay in bed and take a nap when people feel sleepy, but the situations cannot be achieved in the process of daily life and work. Therefore, we often see sleepy people dozing on chairs on buses, subways and planes, which is a convenient way to conserve strength and store up energy. However, when people are sitting on seats, the hips can still be positioned by friction with seat cushions, but the upper body, especially the head, is out of control when people fall asleep. Therefore, when people fall asleep on the seats, people often fall over, thus not only affecting the rest quality, but also causing casualty accidents in serious cases. Therefore, it is particularly important to set up necessary safety precautions on the seats.

### SUMMARY

The purpose of the present disclosure is to provide an auxiliary device for sleeping on a seat. A pillow can be stably installed at the seat headrest. An eye mask and the pillow are encircled to form a ring which can sleeve the human head, and a ring is used to help stabilize the sitting position when the human body is sitting on the seat so that it is difficult for the human body to fall over. The eye mask has the function of isolating external light. Therefore, the rest quality of people is improved, and accidents can also be prevented.

In order to achieve the purpose, the present disclosure provides the following technical schemes. The present disclosure provides an auxiliary device for sleeping on a seat. The auxiliary device for sleeping on a seat includes a pillow. The pillow can be stably connected to a seat headrest through assembly parts. An eye mask is arranged on the side, away from the seat headrest, of the pillow. The eye mask and the pillow are detachably connected through hook-and-loop fasteners. The eye mask and the pillow are encircled to form a ring which can be used for sleeving the head of the human body.

Preferably, the pillow includes a cushion cover and a cushion core filled in the cushion cover.

Preferably, the cushion cover is one of a woven fabric cover, a leather cover and a silicone cover.

Preferably, the cushion core is one of a cotton core, a foam filling core and an inflatable bag.

Preferably, the assembly part includes binding bands and a connecting buckle. Two binding bands are arranged. The connecting buckle includes a female buckle and a male buckle. One end of each of the two binding bands is fixedly connected with the pillow, and the other ends are respectively connected with the female buckle and the male buckle.

Preferably, the female buckle and the male buckle are detachably buckled and connected.

Preferably, the eye mask is made of soft sheets.

Preferably, the convenient engaging mechanisms are hook-and-loop fasteners.

Preferably, the convenient engaging mechanisms are buttons.

Compared with the prior art, the present disclosure has the following beneficial effects.

According to the auxiliary device for sleeping on a seat, the pillow can be stably installed at the seat headrest, the eye mask and the pillow are encircled to form a ring which can sleeve the human head, and the ring is used to help stabilize the sitting position when the human body is sitting on the seat so that it is difficult for the human body to fall over. The eye mask has the function of isolating external light. Therefore, the rest quality of people is improved, and accidents can also be prevented. In addition, the eye mask is detachably connected with the pillow through the hook-and-loop fasteners, and the assembly parts can be quickly disassembled and assembled by using binding bands and connecting buckles, so that the using efficiency of the device is improved.

### BRIEF DESCRIPTION OF THE DRAWINGS

FIG. 1 is a structural schematic diagram of the present disclosure.

FIG. 2 is a structural explosive view of the present disclosure.

Reference signs: 1, pillow; 11, cushion cover; 12, cushion core; 2, eye mask; 3, assembly part; 31, binding band; 32, connecting buckle; 321, female buckle; 322, male buckle; and 4, hook-and-loop fastener.

### DETAILED DESCRIPTION OF THE EMBODIMENTS

The technical schemes according to embodiments of the present disclosure are clearly and completely described in the following with reference to the attached figures in the present disclosure. Apparently, the embodiments in the following description are merely a part rather than all of the embodiments of the present disclosure. Based on the embodiment in the present disclosure, all other embodiments obtained by the ordinary technical staff in the art under the premise of without contributing creative labor belong to the scope protected by the present disclosure.

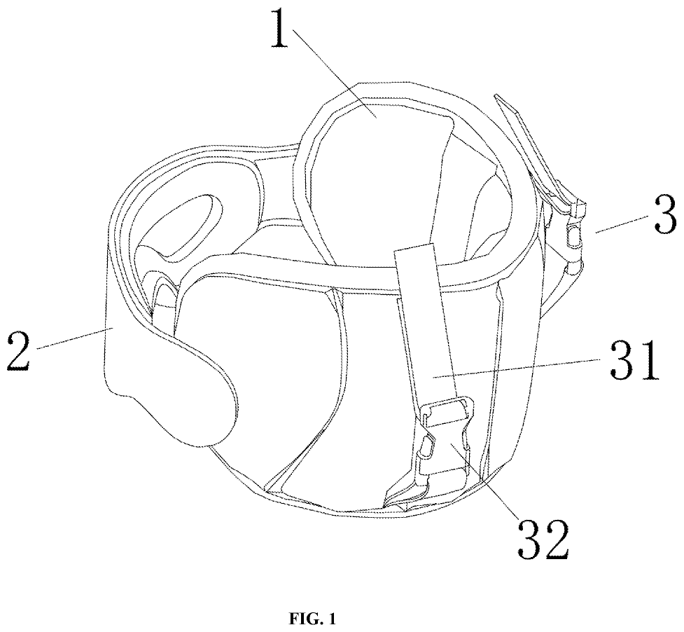

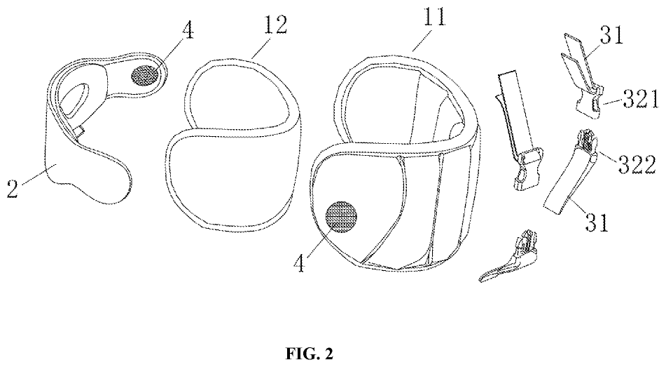

Referring to FIG. 1 and FIG. 2, an auxiliary device for sleeping on a seat includes a pillow 1. The pillow 1 includes a cushion cover 11 and a cushion core 12 filled in the cushion cover 11. The cushion cover 11 is one of a woven fabric cover, a leather cover and a silicone cover. The cushion core 12 is one of a cotton core, a foam filling core and an inflatable bag. The pillow 1 can be stably connected to a seat headrest through assembly parts 3. The assembly part 3 includes binding bands 31 and a connecting buckle 32. Two binding bands 31 are arranged. The connecting buckle 32 includes a female buckle 321 and a male buckle 322. One end of each of the two binding bands 31 is fixedly connected with the pillow 1, and the other ends are respectively connected with the female buckle 321 and the male buckle 322. The female buckle 321 and the male buckle 322 are detachably buckled and connected. During installation, the pillow 1 is placed in front of the seat headrest, then the upper and lower binding bands 21 are respectively wound around the upper and lower edges of the headrest, and the female buckle 321 and the male buckle 322 of the connecting buckle 32 are buckled and connected at the back of the headrest, so that the pillow 1 can be stably installed on the seat headrest through the binding bands. An eye mask 2 is arranged on the side, away from the seat headrest, of the pillow 1. The eye mask 2 and the pillow 1 are detachably connected through convenient engaging mechanisms. The convenient engaging mechanisms can be hook-and-loop fasteners 4 or buttons. In the embodiment, the convenient engaging mechanisms are hook-and-loop fasteners 4. The eye mask 2 is made of soft sheets. The eye mask 2 and the pillow 1 are encircled to form a ring which can be used for sleeving the head of the human body.

In conclusion, according to the auxiliary device for sleeping on a seat, the pillow 1 can be stably installed at the seat headrest, the eye mask 2 and the pillow 1 are encircled to form a ring which can sleeve the human head, and the ring is used to help stabilize the sitting position when the human body is sitting on the seat so that it is difficult for the human body to fall over. The eye mask 2 has the function of isolating external light. Therefore, the rest quality of people is improved, and accidents can also be prevented. In addition, the eye mask 2 is detachably connected with the pillow 1 through the hook-and-loop fasteners 4, and the assembly parts 3 can be quickly disassembled and assembled by using binding bands 21 and connecting buckles 32, so that the using efficiency of the device is improved.

It needs to be noted that in this specification, relational terms such as “first” and “second” are only used to distinguish one entity or operation from another, and do not necessarily require or imply that any actual relationship or sequence exists between these entities or operations. Moreover, the terms “include”, “comprise”, or their any other variants are intended to cover a non-exclusive inclusion, so that a process, a method, an article, or a device that includes a list of elements not only includes those elements but also includes other elements that are not expressly listed, or further includes elements inherent to such a process, method, article, or device.

Although the embodiments of the present disclosure have already been illustrated and described, various changes, modifications, replacements and transformations can be made by those skilled in the art under the condition of without departing from the principle and the spirit of the present disclosure, and thus the scope of the present disclosure should be restricted by claims and equivalents thereof.

What is claimed is:

1. An auxiliary device for sleeping on a seat, comprising a pillow (1) which can be stably connected to a seat headrest through assembly parts (3), wherein an eye mask (2) is arranged on the side, away from the seat headrest, of the pillow (1), the eye mask (2) and the pillow (1) are detachably connected through convenient engaging mechanisms, and the eye mask (2) and the pillow (1) are encircled to form a ring which can be used for sleeving the head of the human body.

2. The auxiliary device for sleeping on a seat according to claim 1, wherein the pillow (1) comprises a cushion cover (11) and a cushion core (12) filled in the cushion cover (11).

3. The auxiliary device for sleeping on a seat according to claim 2, wherein the cushion cover (11) is one of a woven fabric cover, a leather cover and a silicone cover.

4. The auxiliary device for sleeping on a seat according to claim 2, wherein the cushion core (12) is one of a cotton core, a foam filling core and an inflatable bag.

5. The auxiliary device for sleeping on a seat according to claim 1, wherein the assembly part (3) comprises binding bands (31) and a connecting buckle (32), two binding bands (31) are arranged, the connecting buckle (32) comprises a female buckle (321) and a male buckle (322), one end of each of the two binding bands (31) is fixedly connected with the pillow (1), and the other ends are respectively connected with the female buckle (321) and the male buckle (322).

6. The auxiliary device for sleeping on a seat according to claim 5, wherein the female buckle (321) and the male buckle (322) are detachably buckled and connected.

7. The auxiliary device for sleeping on a seat according to claim 1, wherein the eye mask (2) is made of soft sheets.

8. The auxiliary device for sleeping on a seat according to claim 1, wherein the convenient engaging mechanisms are hook-and-loop fasteners (4).

9. The auxiliary device for sleeping on a seat according to claim 1, wherein the convenient engaging mechanisms are buttons.
